(19) United States
(12) Patent Application Publication
(10) Pub. No.: US 2026/0288445 A1
(43) Pub. Date: Sep. 24, 2026

(54) System and Methods of Providing a Software Update to a Medical Device

(71) Applicant: Ventis Medical, Inc., Princeton, NJ (US)

(72) Inventors: Glenn W. Laub, Princeton, NJ (US); David G. Laub, Princeton, NJ (US); Giovanni Meier, Princeton, NJ (US); Bob Gorry, Princeton, NJ (US); Taylor R. Laub, Princeton, NJ (US)

(73) Assignee: Ventis Medical, Inc., Princeton, NJ (US)

(21) Appl. No.: 19/490,119

(22) PCT Filed: Jun. 4, 2024
(86) PCT No.: PCT/US2024/032375

§ 371 Date: Dec. 4, 2025

Related U.S. Application Data
(60) Provisional application No. 63/506,324, filed on Jun. 5, 2023.

(51) Int. Cl. G06F 8/65

(52) U.S. Cl. CPC G06F 8/65

(57) ABSTRACT

A medical device has an electro-mechanical pneumatic assembly configured to generate air flow. The electro-mechanical pneumatic assembly is disposed within the medical device and coupled to one or more components, and a controller is coupled to the electro-mechanical pneumatic assembly. The controller is configured to receive software update data associated with the one or more components, and store the software update data in a memory coupled to the controller. The software update data includes changes to one or more of a parameter of the one or more components, a function of the electro-mechanical pneumatic assembly, and a performance metric of the medical device.

### CROSS-REFERENCE TO RELATED APPLICATIONS

This application claims the benefit of U.S. Provisional Patent Application No. 63/506,324 filed Jun. 5, 2023 entitled “System and Methods of Providing a Software Update to a Medical Device”, which is incorporated by reference herein in its entirety.

### TECHNICAL FIELD

The present disclosure generally relates to a system and method of providing a software update to a medical device and, in some embodiments, to a system and method for providing a software update to a ventilator.

### BRIEF DESCRIPTION OF THE DRAWINGS

The following detailed description of embodiments of the systems and methods of providing a software update to a medical device will be better understood when read in conjunction with the appended drawings of an exemplary embodiment. It should be understood, however, that the invention is not limited to the precise arrangements and instrumentalities shown.

FIG. 1 is a top plan view of a ventilator connected to a patient and having a medical device unit, a breathing circuit, and a patient interface in accordance with an exemplary embodiment of the present invention;

FIG. 2 is a schematic diagram of the ventilator system of FIG. 1;

FIG. 3 is a top perspective view of the medical device unit of FIG. 1;

FIG. 4 is a bottom view of the medical device unit of FIG. 1;

FIG. 5 is a rear perspective view of the medical device unit of FIG. 1;

FIG. 6 is a schematic diagram of the ventilator system of FIG. 1;

FIG. 7 is a network environment configured to allow the ventilator system of FIG. 1 to communicate with one or more external devices; and

FIG. 8 is a flowchart illustrating an exemplary method of using the ventilator system of FIG. 1.

### BACKGROUND

Medical devices, such as ventilators, play a crucial role in modern healthcare, assisting in providing on demand care to patients. These devices often rely on software to function optimally, requiring periodic updates to enhance their performance, security, and compatibility with evolving healthcare standards and regulations.

Traditionally, updating medical device software has been a cumbersome process, often requiring physical access to the medical device by healthcare professionals or software experts. This process can be time-consuming, costly, and disruptive, leading to delays in implementing critical updates and potentially compromising patient care.

### SUMMARY

Embodiments of the present disclosure are directed to a medical device having an electro-mechanical pneumatic assembly configured to generate air flow the electro-mechanical pneumatic assembly disposed within the medical device and coupled to one or more components. The device also includes a controller coupled to the electro-mechanical pneumatic assembly, the controller configured to: receive software update data associated with the one or more components; and store the software update data in a memory coupled to the controller, where the software update data includes changes to one or more of a parameter of the one or more components, a function of the electro-mechanical pneumatic assembly, and a performance metric of the medical device.

In some embodiments, the medical device includes a beacon configured to provide an indication representative of the software update data. The beacon is configured to request the software update. The beacon is configured to determine an availability of the software update. The beacon transmits a software version of the medical device to a server on a periodic basis and the server compares the software version to the software update and transmits the software update to the medical device.

In some embodiments, the controller receives the software update without human intervention. The controller is configured to output a signal to one or more of a speaker, a user interface, a storage device, an external device, a beacon, a writing device, a transmitting device, and an indicator.

In some embodiments, the medical device includes a wake-up controller coupled to the controller, the wake-up controller configured to power on the medical device prior to the controller receiving the software update data. The controller is further configured to display on a user interface, via a display screen or a light indicator, a software status associated with the software update data. The controller includes a low power controller configured to receive the software update data.

In some embodiments, the controller is configured to receive plurality of inputs from a user, the plurality of inputs associated with operation of the medical device, store the plurality of inputs in a database, and receive subsequent software update data including instruction data associated with the plurality of inputs. The controller is further configured to receive, from a user, a plurality of audio prompts; use one or more models, parse and extract from the audio prompts one or more keywords associated with a request, and output an audio response based on the request.

Another embodiment of the present disclosure may provide a method of providing a software update to a medical device. The method includes receiving, software update data associated with one or more components of a ventilator, the ventilator including an electro-mechanical pneumatic assembly disposed within the ventilator and coupled to the one or more components. The method also includes storing the software update data in a memory coupled to a controller, the controller coupled to the electro-mechanical pneumatic assembly. The method also includes where the software update data includes changes to a parameter of the one or more components.

In some embodiments, the method includes receiving a plurality of inputs from a user, the plurality of inputs associated with operation of the medical device, storing the plurality of inputs in a database, and receiving subsequent software update data including instruction data associated with the plurality of inputs.

In some embodiments, the method includes receiving, from a user, a plurality of audio prompts, using one or more models, parse and extract from the audio prompts one or more keywords associated with a request, and outputting an audio response based on the request.

In some embodiments, the method includes powering on the medical device, via a wake-up controller coupled to the controller, prior to the controller receiving the software update data. The method may include displaying on a user interface, via a display screen or a light indicator, a software status associated with the software update data.

In some embodiments, the method includes determining that the medical device has transitioned from use state to a non-use state, and retrieving the software update data from the memory for installation in response to the determination that the medical device is the non-use state.

Another embodiment of the present disclosure may provide a method of providing a software update to a medical device. The method includes receiving software update data associated with a ventilator, the ventilator including an electro-mechanical pneumatic assembly coupled to one or more components. The method also includes storing the software update data in a memory coupled to a controller, the controller coupled to the electro-mechanical pneumatic assembly. The method also includes determining that the ventilator is in a ventilation state, the ventilation state being when the electro-mechanical pneumatic assembly is providing ventilation to a patient. The method also includes preventing retrieval of the software update data from the memory for installation in response to the determination that the ventilator is in the ventilation state.

In some embodiments, the method includes determining that the ventilator has transitioned from the ventilation state to a non-ventilation state, and retrieving the software update data from the memory for installation in response to the determination that the ventilator is the non-ventilation state.

### DETAILED DESCRIPTION

Medical devices, such as ventilators, are commonly used for providing respiratory therapy and/or assistance to patients in respiratory distress. Most ventilators include many components working together to provide adequate breaths to a patient. During use or storage, the software controlling the ventilator can fail or be out of date resulting in a ventilator no longer operating correctly and becoming defective. An out-of-date ventilator may not be identified prior to when it needs to be used, especially if the ventilator is stored with many other ventilators in a storage unit or a stockpile. Further, consistent testing and interaction with the ventilators to ensure that they are up to date is time consuming.

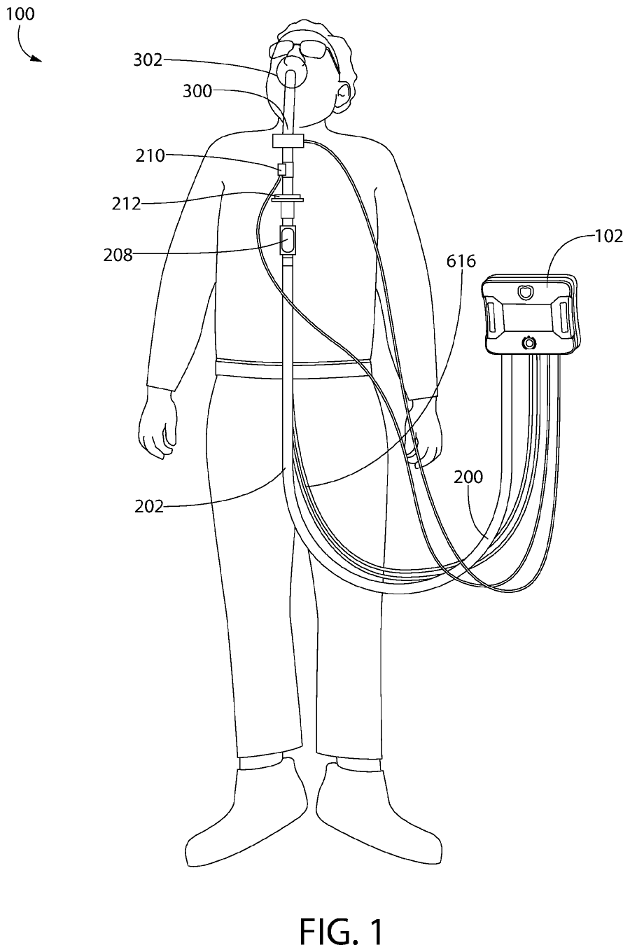

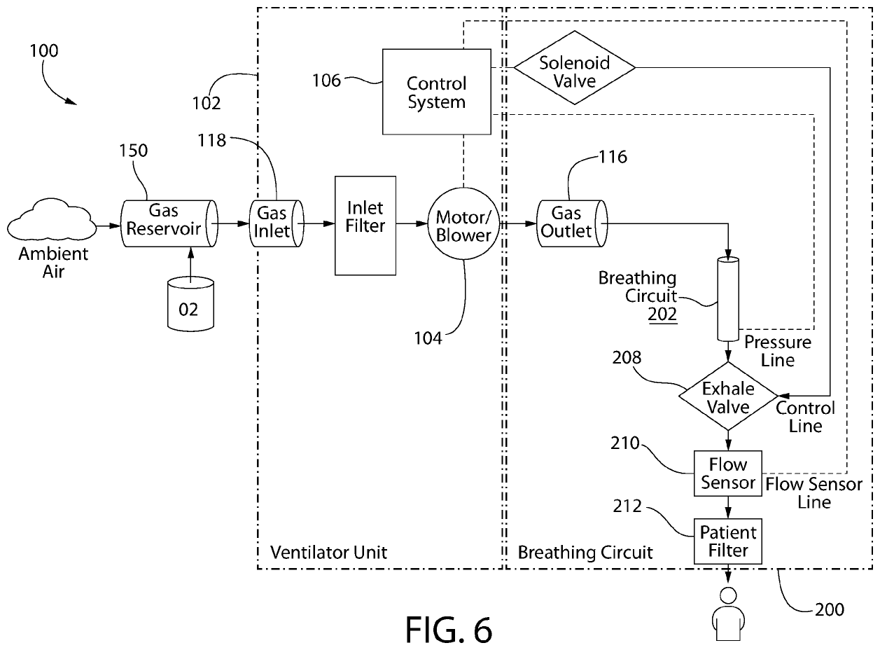

Exemplary embodiments of the present invention provide a system and method of providing a software update to a medical device. Embodiments of the present invention provide a system (e.g., ventilator system), generally designed 100, as shown in FIGS. 1-6. In use, ventilator system 100, which may also be referred to herein as simply a ventilator (e.g., ventilator 100), may be used to provide assistance with breathing for the treatment of patients in a medical setting, such as the intensive care unit (ICU) of a hospital or a medical clinic. Ventilator 100 may also be used in other settings such as an ambulance, ambulatory center, in/out-patient centers, nursing homes/long term care facilities, and mobile clinics that can go to a patient directly. In some embodiments, ventilator 100 is portable to allow for use in different environments. For example, ventilator 100 may be easily transportable to be used in mobile settings (e.g., an ambulance). In some embodiments, ventilator 100 includes a medical device unit being compact in size to allow for portability. For example, the medical device unit (e.g., ventilator) of ventilator 100 may include a single circuit board containing all necessary or a majority of the critical control components, and desired components to reduce the overall footprint of the medical device unit.

In some embodiments, ventilator 100 allows for rapid initiation of emergency ventilation to a patient in respiratory distress. Ventilator 100 may be a rescue ready system configured to provide emergency treatment (e.g., ventilation) to a user or individual in respiratory distress. Ventilator 100 may be configured to provide rapid, emergency ventilation to a patient with minimal to no leakage of waste of air. Ventilator 100 may provide an efficient system for providing ventilation to a patient.

In use, ventilator 100 may be used for the treatment of patients in a medical setting. For example, ventilator 100 may be a ventilator to assist patients in respiratory distress or acute respiratory failure. Ventilator 100 may include medical device unit 102, breathing circuit 200, and patient interface 300. Medical device unit 102 may be configured to provide mechanical ventilation to a patient under respiratory failure through breathing circuit 200. Medical device unit 102 may provide the necessary gas flow or airflow, which may be directed through breathing circuit 200 to patient interface 300, which is coupled to the face of a patient. Medical device unit 102 may include blower 104, control system or controller 106 and power supply 108. Breathing circuit 200 may include tube 202 which may be coupled to medical device unit 102 at first end 204 and coupled to patient interface 300 at second end 206.

In some embodiments, medical device unit 102 may be a ventilator used to provide assistance to a patient in respiratory distress. Medical device unit 102 may be configured to provide different modes of ventilation to a patient. For example, medical device unit 102 may be configured to provide assist-controlled ventilation, volume-controlled ventilation, pressure support, pressure-controlled ventilation, pressure regulated volume control, positive end expiatory pressure, synchronized intermittent-mandatory ventilation, and/or manual ventilation. Medical device unit 102 may be used instead of a bag valve device, an emergency transport ventilator, or any other modes or devices for providing ventilation to a patient.

Medical device unit 102 may include software or firmware configured to control medical device unit 102. For example, medical device unit 102 may include memory (e.g., memory 115) configured to store software to that provide instructions to a processor or control unit (e.g., control system 106). In some embodiments, the software by be routinely updated. For example, the software of medical device unit 102 may need to be updated to add additional features, improve security, or fix issues with medical device unit 102.

In some embodiments, the software or firmware is stored on an SOM (system on module) and/or a microcontroller. For example, medical device unit 102 may include one or more SOMs and/or microcontroller, which may include software for controlling the functions and/or one more components of medical device unit 102. The SOM and/or microcontroller may also include an operating software. In some embodiments, changes (e.g., updates) to the software or firmware of medical device unit 102 includes changes to the operating software.

Medical device unit 102 may receive a software update data package (“software update”), download the software update, and install the update. For example, medical device unit 102 may receive the software update pushed (e.g., transmitted) to it via Bluetooth, WiFi, Near Field Communication (NFC)) or via a wired connection. In some embodiments, the software update is encrypted and verified prior to flashing (e.g., updating) to the read-only memory (ROM) of the SOM and/or microcontroller. In some embodiments, the software of the SOM and/or microcontroller includes a boot loader configured to update and/or execute the software update. In some embodiments, medical device unit 102 is configured to download the software update prior to installing the software update. For example, an external device may be used to push (e.g., wirelessly transmit) the software update to medical device unit 102, which may download the software prior to installing. In some embodiments, medical device unit 102 downloads the entirety of the software update prior to installing. Alternatively, medical device unit 102 may download and install the software update in segments. For example, medical device unit 102 may download a segment of the software update, install the segment of the software update, download a subsequent segment of the software update, and then install the segment portion of the software update. In some embodiments, an external device proximate medical device unit 102 automatically sends a software update that medical device unit 102 downloads while the external device is proximate medical device unit 102. An external device may be proximate to a plurality of medical device units 102 and the plurality of medical device units 102 may each be configured to download a software update pushed from the external device.

In some embodiments, medical device unit 102 is configured to revert to a prior software version. For example, medical device unit 102 may receive a software update, but the installation of the software update may fail. Medical device unit 102 may revert to a the most recent previous working version to ensure that medical device unit 102 remains operable. In some embodiments, the software update includes two or more versions. For example, a software update may include software A and software B such that if software A fails (e.g., during downloading or installation), software B may proceed and allow medical device unit 102 to be used. In some embodiments, the difference between software A and software B are minor. For example, software A may include one or more minor features not present in software B.

In some embodiments, medical device unit 102 includes a medical WiFi adapter (“WiFi adapter”) configured to independently receive software updates over a WiFi connection without endangering critical function of medical device unit 102. Medical device unit 102 may receive updates via over-the-air (OTA) updates. In some embodiments, medical device unit 102 is configured to receive OTA updates via cellular signal. For example, medical device unit 102 may be configured to communicate with a server over the cellular network and may receive software updates (e.g., OTA updates) over the cellular network when WiFi is unavailable. In some embodiments, the software or firmware for medical device unit 102 may be updated wirelessly (e.g., over-the-air updates, Bluetooth, WiFi, Near Field Communication (NFC)) or via a wired connection. The software of medical device unit 102 may be updated periodically or aperiodically. In some embodiments, medical device unit 102 is configured to have updates pushed to it from a central server or central database and medical device unit 102 is configured to install the updates.

In some embodiments, the software update includes updates to the operating system (e.g., Linux OS, Windows OS, macOS), the application code, and various resource files. The application code can add, repair or enhance features in the performance of medical device unit 102, which can impact patient care. The updates may impact the user experience by, for example, adding additional parameters and graphics to the GUI (e.g., user interface 124) or additional languages (e.g., English, Spanish, Japanese, etc.).

In some embodiments, medical device unit 102 is configured to download and install software updates based on the location of medical device unit 102 (e.g., geofencing). For example, medical device unit 102 may include a GPS receiver configured to transmit the location of medical device unit 102. Medical device unit 102 may receive software updates when medical device unit 102 is in storage or not in use. In other words, medical device unit 102 may be configured to download and install a software update when the GPS receiver is located within a safe zone.

In some embodiments, medical device unit 102 is susceptible to tampering or hacking of wireless communication and thus is configured to activate and deactivate its wireless communication modalities. Over-the-air updates or remote software updates may be enabled in the safe zone to minimize the risk of tampering or hacking of wireless communication. This risk can be partially mitigated by only allowing over-the-air updates to medical device unit 102 to be performed inside a safe zone associated with the GPS coordinates of medical device unit 102. In alternative embodiments a user can define a safe zone within a hospital or secure setting that also allows over-the-air updates to software or firmware for medical device unit 102 to be installed. In some embodiments, to further ensure integrity of any updates upon entering the safe zone a user is required to perform an authentication before having to manually accept an over-the-air update.

Medical device unit 102 may be configured to update its software when stored or in storage, during start-up, or during use. Prior to a software update, a control system of medical device unit 102 may transmit one or more signals to various components of medical device unit 102 to determine whether they are operating correctly. For example, medical device unit 102 may cause one or more components of medical device unit 102 to go through calibrations and checks to ensure that they are performing optimally or within specific parameters based on the desired use of medical device unit 102. In response to a component not performing within parameters, medical device unit 102 may transmit a status update to an external device and medical device unit 102 may receive a software update in response.

In some embodiments, medical device unit 102 performs a software update upon powering on. Medical device unit 102 may perform a software update automatically without being requested to do perform a software update. In some embodiments, medical device unit 102 is configured to perform a software update without human intervention. However, medical device unit 102 may be configured to perform a software update upon a request received from a remote device or server. The software update may be configured to modify or improve the user interface, the functioning of medical device unit 102, or other parameters of medical device unit 102 (e.g., thresholds for sensor, calibrations, timing protocols, etc.).

In some embodiments, the software update is configured to add additional functionality to medical device unit 102. For example, the software updated may modify the existing software or firmware to add additional ventilation modes or change the ranges/thresholds of the modes. For example, the software update may increase the range of tidal volume (TV) for inclusion of infants and children. The software update may provide non-invasive ventilation and/or add AI (artificial intelligence) optimization of ventilation so that medical device unit 102 can adjust/optimize the ventilation parameters to a patient's specific clinical condition and respond to their changing clinical condition. The software update may add additional languages, adding new tools to improve the graphical user interface (e.g., user interface 124) such as zoom, pan of data, brightness, saturation, contrast, etc. In some embodiments, the software update is configured to change default ventilation parameters for specific applications. For example, medical device unit 102 may be used in a military setting, which may require default different settings for default weight/size than, for example, a pediatric hospital. As one or more components of medical device unit 102 age and additional post market information and data becomes available, the parameters, thresholds, and/or calibration requirements for one or more components of medical device unit 102 may be required to be changed via one or more software updates.

In some embodiments, medical device unit 102 is configured to perform a software update upon passive interrogation. For example, medical device unit 102 may receive a software update without sending a request. In some embodiments, medical device unit 102 is configured to perform a self-test to test various components of medical device unit 102. The results of the self-test may be used to determine what software updates need to be installed or applied to the various components of medical device unit 102.

In some embodiments, medical device unit 102 is configured to perform a software update of one or more components of medical device unit 102 upon initial start-up (e.g., boot-up) and/or upon restarting of medical device unit 102. The same components of medical device unit 102 may be tested during start-up and restart. However, medical device unit 102 may perform software update son different components based on whether medical device unit 102 is starting up or restarting. For example, upon start-up, medical device unit 102 may perform a software update on a first set of components. Upon restart, medical device unit 102 may perform a software update on a second set of components. The first set of components and the second set of components may be different, the same, or include overlapping components.

In some embodiments, medical device unit 102 includes a button that when actuated causes medical device unit 102 to perform a software update. In some embodiments, medical device unit 102 is configured to perform a software update without coupling to any other device. For example, in use medical device unit 102 may couple to patient interface 300 via breathing circuit 200. Medical device unit 102 may be configured to perform a software update prior to coupling to breathing circuit 200. In some embodiments, medical device unit 102 is configured to perform a software update without having to couple to breathing circuit 200 and/or patient interface 300.

Referring to FIGS. 1-5, medical device unit 102 may include housing 132, blower 104, control system 106, and power supply 108. Housing 132 of medical device unit 102 may house and protect the components disposed within medical device unit 102. Housing 132 may be lightweight to allow for easy portability of medical device unit 102. For example, housing 132 of medical device unit 102 may be made of a lightweight polymer to allow for easy transportation. In some embodiments, housing 132 is comprised of one or more of acrylonitrile butadiene styrene acrylonitrile butadiene styrene (ABS), polyoxymethylene (POM), aliphatic polyamides (PPA), polycarbonate (PC), polyphenylsulfone (PPSU), polyetherimide (PEI), and polypropylene (PP). Housing 132 may be comprised of a lightweight, but durable material to allow for repeated use in harsh environments, while still providing portability. For example, housing 132 may be comprised of ABS to provide portability and to ensure that the components disposed within housing 132 are secured, protected, and remain undamaged. In some embodiments, housing 132 of medical device unit 102 is substantially rectangular shaped to allow for easy storage. However, housing 132 may be square, circular, triangle, octagonal, or any other shape desired. In some embodiments, housing 132 includes sidewalls 130. In a preferred embodiment, housing 132 includes four sidewalls 130 to define a substantially rectangular shape of medical device unit 102. In some embodiments, housing 132 has rounded corners and beveled edges to allow for a more ergonomic shape.

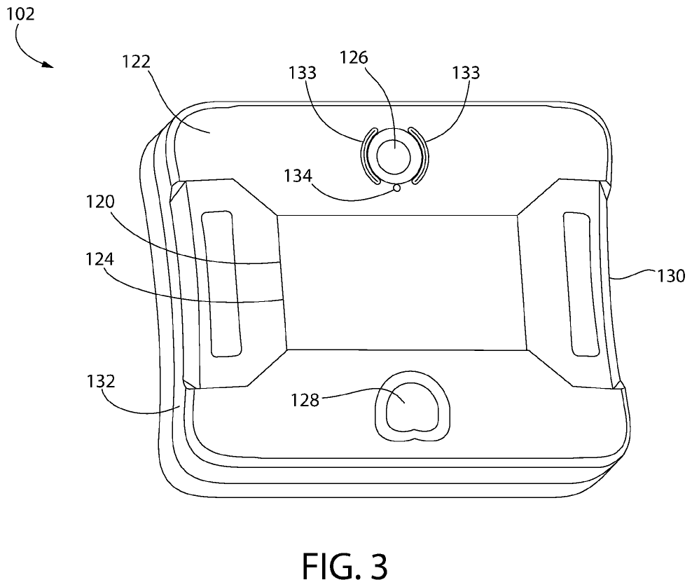

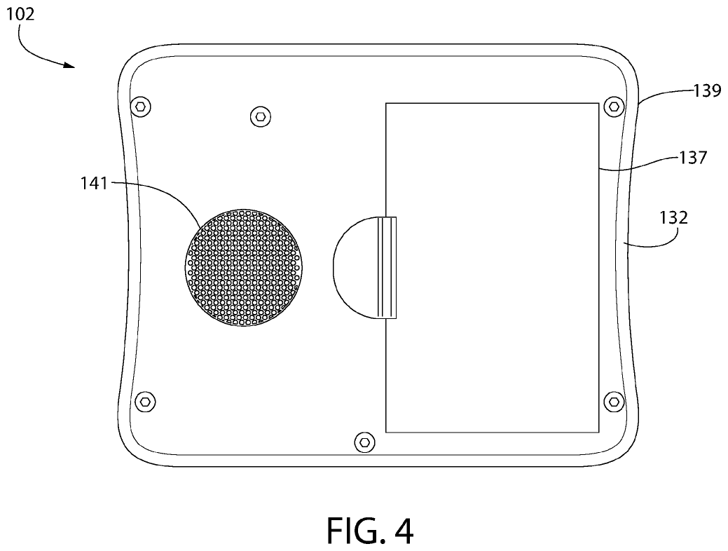

Referring to FIGS. 3-4, housing 132 may include top surface 122 and bottom surface 139. In some embodiments, top surface 122 is parallel to bottom surface 139. Top surface 122 may be coupled to bottom surface 139 via sidewalls 130. Housing 132 may include cutout 120 disposed on top surface 122 of housing 132. Cutout 120 maybe sized and shaped to receive user interface 124. User interface 124 may be a display device, which may be disposed within cutout 120, and may be configured to receive input from a user. In some embodiments, user interface 124 is a graphical user interface. For example, user interface 124 may be a touch screen configured to receive inputs from a user and transmits the inputs to control system 106. Further, user interface 124 may be used to display information about a patient using medical device unit 102. For example, user interface 124 may display an indication of the respiratory status of a patient coupled to patient interface 300.

In some embodiments, user interface 124 may display various settings, parameters, and/or functionalities of the components disposed within medical device unit 102. For example, user interface 124 may display the peak inspiratory pressure (PIP), tidal volume (TV), respiratory rate (RR), positive end expiratory pressure (PEEP), inspiratory to expiatory ratio (I:E ratio), ventilation mode, peak flow, and sensitivity. User interface 124 may be coupled to control system 106 and may be configured to control various components of ventilator 100. For example, a user may interact with user interface 124 to change parameters of blower 104. In some embodiments, medical device unit 102 receiving software updates results in modification to the visual representations of user interface 124.

In some embodiments, medical device unit 102 includes speaker 141. Speaker 141 and/or user interface 124 may be configured to provide instructions and/or alerts to the user. For example, user interface 124 may provide visual instructions to a user for correcting an error to medical device unit 102 and speaker 141 may provide audio instructions to a user for correcting an error to medical device unit 102. Medical device unit 102 may utilizes natural language processing (NLP) to receive audio prompts from a user and generate audio instructions. For example, medical device unit 102 may include a smart assistant (e.g., digital assistant) utilizing NLP. In some embodiments, the smart assistant is implemented within medical device unit 102 to receive prompts from the user and provide instructions/guidance to the user. As discussed below, medical device unit 102 may utilize one or more models to provide instructions to a user.

In some embodiments, user interface 124 is configured to display a video or graphics to a user to instruct them on how to fix or address an error to medical device unit 102. In some embodiments, speaker 141 is configured to provide an audio alert or alarm to a user based on an error detected by medical device unit 102. User interface 124 may be configured to provide a visual alert or alarm to a user based on an error detected by medical device unit 102. In some embodiments, medical device unit 102 includes a vibrator such that when an error occurs, medical device unit 102 vibrates or provides other haptic feedbacks. Medical device unit 102 may transmit an outgoing signal when an alert occurs to alert a remote user.

In some embodiments, a user interacts with user interface 124 to change various operating modes and/or parameters of medical device unit 102. For example, user interface 124 may provide an option for adjusting the PEEP, the PIP, the tidal volume, the I:E ratio, or other parameters.

In some embodiments, medical device unit 102 includes beacon or indicator 134 to provide a status of ventilator 100. Indicator 134 may provide the status of ventilator 100 and/or medical device unit 102. For example, indicator 134 may indicate whether medical device unit 102 is damaged, inoperable, and/or functionally properly. Indicator 134 may be an LED and control system 106 may transmit a status to indicator 134 causing indicator 134 to illuminate a specific color and flash at specific frequency. For example, indicator 134 may be a visual indicator, such as an LED, indicating that a software update has been received and installed by medical device unit 102. In some embodiments, indicator 134 displays a first condition (e.g., a first color) if a software update has been received and displays a second condition (e.g., a second color) if the software update has been installed. In some embodiments, indicator 134 continuously provides a visual indication that medical device unit 102 is functioning properly and an interruption in the visual indication indicates that an error has occurred with medical device unit 102 or that a software update is required.

In some embodiments, medical device unit 102 receives specific updates based on the error that has occurred. For example, software updates may be sent to a specific medical device unit 102 based on the error received by the specific medical device unit 102. Software updates may be pushed and/or transmitted to medical device unit 102 based on errors received by a server associated with a status check or self-test performed by medical device unit 102. For example, a threshold and/or parameter may be adjusted using a software update for a specific medical device unit 102 to improve performance or resolve an issue. Alternatively, the software update to adjust the threshold and/or parameter may be sent to multiple medical device units 102 even though a subset of medical device units 102 encounter an error.

In some embodiments, medical device unit 102 includes a beacon in addition to or alternative to indicator 134. The beacon may be configured to transmit a signal (e.g., wireless signal) regarding the status of medical device unit 102. The beacon may be configured to transmit, periodically or aperiodically, the current software version of medical device unit 102 to a server.

The server may compare the software version to the version of the software update and if the software version does not match the version of the software update, the server may push the software update to medical device unit 102.

In some embodiments, indicator 134 provides a status of medical device unit 102 without requiring a user to interact with or power on medical device unit. For example, indicator 134 may be coupled to a power supply separate from power supply 108 and may be configured to illuminate to provide an indication of a status to a user without the user interacting with medical device unit 102. Indicator 134 may transmit a signal to an external receiving device. In some embodiments, indicator 134 transmits a signal regardless of whether an external receiving device is proximate to medical device unit 102 or whether an external receiving device is requesting data from indicator 134. For example, indicator 134 may be configured to transmit a signal regardless of whether a device is listening or whether a device is requesting a signal from indicator 134. In some embodiments, indicator 134 is configured to always be transmitting a signal when medical device unit 102 is functioning correctly or operating normally.

In some embodiments, indicator 134 is configured to flash different colors of light. For example, indicator 134 may flash the color green when medical device unit 102 is operating normally, flash the color red when medical device unit 102 is malfunctioning or requires a software update, or flash the color yellow when medical device unit 102 has an error, but can still function. However, indicator 134 may flash or have a constant illumination. Indicator 134 may be any color desired and may alternate between different colors depending on the status of medical device unit 102. In some embodiments, indicator 134 is coupled to a power supply to ensure that indicator 134 is able to continuously provide an indication for the status of medical device unit 102.

In practice, control system 106 may perform a software update without user intervention and may cause indicator 134 to illuminate based on the completion of the software update (e.g., green) or an error (e.g., red) that occurred during the software update. A user may view medical device unit 102 after the software update has been performed and may view indicator 134. Upon viewing indicator 134, a user may be able to determine if the software update was successful and if there are any errors associated with medical device unit 102 without having to interact with medical device unit 102. Interacting with medical device unit 102 may including actuating one or more buttons on medical device unit 102, powering on medical device unit 102, or engaging with user interface 124. In practice, a user may view indicator 134 immediately after the software update has been performed or may view indicator 134 after a duration of time as elapsed since the software update has been performed.

Medical device unit 102 may include one or more buttons that control ventilator 100. For example, medical device unit 102 may include buttons 126 and 128, which control the power status and functions of medical device unit 102. In some embodiments, button 126 is a power ON/OFF button to control the power status of medical device unit 102. For example, a user may press button 126 to power on medical device unit 102. Button 128 may be a manual breath button, which delivers a single breath at a predetermined tidal volume to a patient. In some embodiments, button 128 may need to be pressed for a predetermined amount of time before medical device unit 102 delivers a single breath to the patient.

Referring to FIGS. 1-5, medical device unit 102 may include pneumatic assembly or blower 104, which may include motor 110 and fan 112. Pneumatic assembly 104 may be an electro-mechanical pneumatic assembly or system. Motor 110 may be coupled to fan 112 and motor 110 may be configured to rotate fan 112 to generate air flow. In some embodiments, motor 110 is configured to rotate fan 112 at maximum of 37,500 revolutions per minute (RPM). Fan 112 may rotate to generate airflow that exits blower 104. Motor 110 may be coupled to control system 106, which may control motor 110. In some embodiments, fan 112 is configured to provide a maximum of 1,000 liters per minute (LPM). In some embodiments, fan 112 is configured to rotate at greater than 37,500 RPMs and greater than 1,000 LPMs.

Blower 104 (e.g., electro-mechanical pneumatic assembly) may be configured to receive a software update via control system 106. For example, blower 104 may be coupled to control system 106, which may be configured to control the activation and flow rate of blower 104. Control system 106 may include a software update directed to blower 104. In some embodiments, the software update changes parameters of blower 104 (e.g., flow rate, activation thresholds, duration of activation, direction of flow). For example, the ramping up and/or down of blower 104 may be modified during a software update based on performance characteristics or the history of blower 104 (e.g., via a status update/check or self-test).

In some embodiments, blower 104 may be disposed within enclosure 114. Enclosure 114 may be sized and shaped to receive blower 104 and may be a unitary piece. For example, enclosure 114 may be comprised of two halves and may be configured to receive blower 104 such that blower 104 is disposed within enclosure 114. Enclosure 114 being made comprised of two halves which surround blower 104 allows for the reduction of components and material needed to manufacture ventilator 100. Blower 104 may include an inflow, which may be disposed within enclosure 114. In some embodiments, the inflow of blower 104 may be the only portion of blower 104 disposed within enclosure 114.

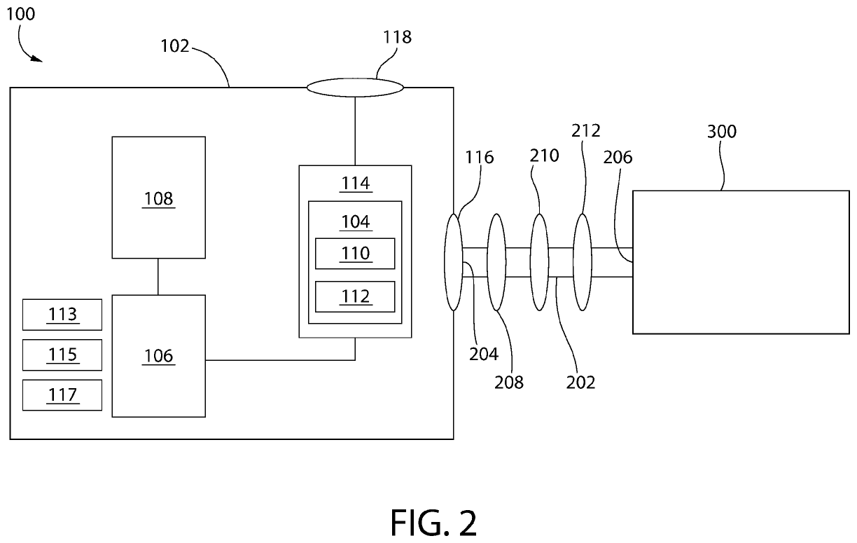

Referring to FIGS. 1-2 medical device unit 102 may include control system 106. Control system 106 may be a microcontroller, a peripheral interface controller (PIC), a system on a chip (SoC), or a processor. In some embodiments, control system 106 is a low power controller. For example, control system 106 may be a low power controller coupled to a power supply such that control system 106 is configured to run for extended period of time (e.g., several years). Control system 106 may be coupled to one or more components of ventilator 100. In some embodiments, control system 106 is coupled to blower 104 to control motor 110, which controls fan 112. In some embodiments, control system 106 controls the volume of gas delivered to a patient by attenuating the speed of fan 112. For example, controls system 106 may attenuate the power delivered to motor 110, thereby decreasing the speed of fan 112 to reach a target amount of gas delivered to a patient through breathing circuit 200. Control system 106 may include writing device 113, which may be configured to write information to transmitting devices 117, such as radio-frequency identification (RFID) chips/tags. In some embodiments, control system 106 is coupled to power supply 108. However, control system 106 may be coupled to its own power supply. Control system 106 may include software configured to control system 106 and one or more components coupled to control system 106. The software may be updated from a remote server or external device.

In some embodiments, the software of control system 106 is updated wirelessly. For example, control system 106 may receive a software update for software providing instructions to control system 106 and/or other components of ventilator 100. In some embodiments, medical device unit 102 includes an encryption engine to allow medical device unit 102 to transmit and receive communication (e.g., software updates, software status, status of medical device unit 102). The encryption engine may be stored within memory 115 and configured to encrypt any transmissions and/or communications prior to delivering the transmissions and/or communications to a server or other device. The encryption engine may use a lightweight encryption algorithm, which allows for quick and efficient decryption by medical device unit 102. Control system 106 may not have significant processing power, therefore the lightweight encryption algorithm allows medical device unit 102 to decrypt the transmission and/or communication faster and also requires less processing power for decryption.

In some embodiments, an external device (e.g., a server) communicates with medical device unit 102 (e.g., control system 106) via the encryption engine. Medical device unit 102 may store information, such as the software update data package within a trusted memory (e.g., memory 115) that is not in communication with other devices. The trusted memory ensures that the software update resides within a clean environment, i.e., an environment free from malware, spyware, viruses, root kits or other vulnerabilities. In some embodiments, control system 106 sends any and all communications, commands and/or instructions to the encryption engine prior to transmission and/or communication to external devices (e.g., server). The encryption engine, which resides within the trusted memory, encrypts the transmission and/or communication prior to sending the communication to an external device via WiFi or a cellular network.

In some embodiments, the external device transmitting or pushing the software update pairs the software update with an authentication token. The encryption engine may be configured to authenticate the authentication token to ensure that the software update came from a trusted source. For example, the external device (e.g., server) may interact with medical device unit 102 to provide to medical device unit 102 one or more authentication factors, such as a token, to authenticate the external device. The external device may transmit an authentication token to medical device unit 102. In some embodiments, the one or more authentication factors are configured to verify, authenticate or authorize communication between the external device and medical device unit 102. After verification, authentication or authorization of the external device with medical device unit 102, the external device and/or medical device unit 102 may establish a secure connection.

The external device may obtain one or more authentication factors, which may include a certificate, a password, an authentication token and/or a cloud authentication token. The password may be provisioned at manufacturing, fabrication, packaging or distribution of medical device unit 102 and may contain any number of alphanumeric characters of any length, such as a password length of 5 alphanumeric characters or 20 bits. The password may be written within a packaging or within a manual of medical device unit 102, such that a user of medical device unit 102 and/or the external device has access to the password. For example, medical device unit 102 may receive via user input through the user interface 124 a password associated with the software update transmitted by the external device. In some embodiments, the software update received by medical device unit 102 may be encrypted using tokens, keys, blockchain, passwords, hashes, or other encrypting methodologies. In some embodiments, the custody chain of medical device unit 102 and/or the software updates transmitted to and installed by medical device unit 102 are managed and tracked using blockchain. For example, blockchain technology may be used create applications to track the ownership and possession of one or more medical device units 102 to securely track and determine the chain of custody.

In some embodiments, the server is configured to encrypt the software update with an encryption key prior to transmitting the software update to medical device unit 102. The encryption engine may be configured to decrypt the software update upon downloading the software update. In some embodiments, the server is configured to determine whether medical device unit 102 is in a secured setting (e.g., in secure communication with the server) and thus there is no need for the server to encrypt the software update package.

In some embodiments, the server is configured to poll medical device unit 102 on a periodic or aperiodic basis. Medical device unit 102, in response to the polling, may transmit status data indicating that there is an error and/or that a status update is required. In some embodiments, the server is configured to poll for medical device unit 102 on a periodic or aperiodic basis and respond if appropriate. Alternatively, medical device unit 102 may transmit a signal on a periodic or aperiodic basis and the server may respond (e.g., transmitting a software update). A user may also initiate a request for a software update manually, or medical device unit 102 may poll the server. In some embodiments, a user receives the software update from the server to an electronic device (e.g., mobile phone, laptop, desktop, tablet, etc.) and transmits the software update from the c electronic device to medical device unit 102. For example, a user, via the electronic device, may download the software update and transmit it to medical device unit 102. In some embodiments, a user downloads the software update to their electronic device and transmits, either wirelessly or via a wired connection, to medical device unit 102. The electronic device may transmit the software update to a plurality of medical device units 102. For example, the electronic device may be configured to transmit the software update to a plurality of medical device units 102 automatically when each medical device unit 102 is in proximity to the electronic device. In some embodiments, a drone or UAV may be used to wirelessly transmit the software update to one or more medical device units 102 when in proximity to the drone or UAV. Medical device unit 102 may receive the software update via hotspotting, Bluetooth, WiFi, NFC, or other communication modalities.,

In some embodiments, medical device unit 102 is configured to power on (e.g., via a self-test or a user) and poll the server to determine whether a software update is required. The server may be configured to transmit wake-up or power on commands to medical device unit 102 to cause medical device unit 102 to power on. Upon powering on, the server may transmit the software update to medical device unit 102 for downloading and installing.

In some embodiments, the server keeps the transmission of the software upgrade and the authentication token/key as separate data package to make hacking or interception more difficult. In some embodiments, the server generates a server private key uses the server private key to digitally sign the software update. The encryption engine may be configured to recognize the server private key and determine that the software update came from a trusted source. In some embodiments, after the server encrypts the software update using the authentication key/token, the server establishes a connection with control system 106 to transmit the software update to medical device unit 102. Once the server has encrypted the software update and has established the connection with control system 106 of medical device unit 102, the server may send, provide, and/or transmit the encrypted software update to control system 106. In some embodiments, based on a self-test, medical device unit 102 determines that that a software update is necessary and obtains the encrypted software update from the server.

In some embodiments, control system 106 does not have access to the contents of the software update until control system 106 decrypts the software update. For example, the server may encrypt the software update using an authentication token/key and may transmit the encrypted software update to control system 106 of medical device unit 102. The encryption engine of control system 106 may be configured to decrypt the encrypted software update to access the contents and install the software update. In some embodiments, encryption of the software data is FDA and HIPAA compliant.

In some embodiments, the software update does not include patient identifiable information or includes de-identified patient information. During a software update, de-identified patient and unit information might be sent to the server confirming the status of the software update status, status of medical device unit 102, and/or performance of medical device unit 102.

In some embodiments, writing device 113 is disposed within medical device unit 102. However, writing device 113 may be disposed outside of medical device unit 102 and may be an external device. Writing device 113 may be disposed within, on, or outside of medical device unit 102 and may wirelessly communicate with transmitting device 117. In some embodiments, writing device 113 is configured to wirelessly write information to transmitting devices 117. Writing device 113 may be coupled to control system 106 and may be stored anywhere within medical device unit 102. Writing device 113 may further be coupled to memory 115, which may be coupled to control system 106.

In some embodiments, transmitting device 117 is stored within medical device unit 102 and is communicatively coupled to control system 106. However, transmitting device 117 may be disposed on or near housing 132 of medical device unit 102 and may be configured to wirelessly communicate with control system 106. For example, transmitting device 117 may be coupled to the exterior surface of housing 132 and may wirelessly receive information from control system 106. Transmitting device 117 may be a storage device configured to wirelessly transmit information, such as a wireless transmitting device. For example, transmitting device 117 may include one or more of an RFID chip/tag, a near-field communication chip, a Bluetooth transmitter, a digital barcode, QR code, or a WiFi module. In some embodiments, user interface 124 is configured to display the digital barcode or QR code such that a user can scan the digital barcode or QR code to retrieve status date of one or more medical device unit 102. In some embodiments, transmitting device 117 transmits information upon request. However, transmitting device 117 may be configured to transmit information associated with a self-test automatically and/or autonomously without intervention by a user or external device, or without receiving a request to transmit information. Transmitting device 117 may be configured for low-power consumption. In some embodiments, transmitting device 117 is configured to receive power only from an external source. However, transmitting device 117 may be powered by power supply 108 or its own power supply.

Control system 106 may receive information associated with, for example, the status of ventilator 100 and store the information in memory 115 or directly to transmitting device 117. Writing device 113 may access memory 115 and may write the information stored within memory 115 to transmitting device 117. In some embodiments, memory 115 includes transmitting device 117. Memory 115 may include, for example, random access memory (RAM), a hard disk drive and/or a removable storage drive, such as a floppy disk drive, a magnetic tape drive, an optical disk drive, or a wireless device, such as an RFID tag. Memory 115 may include other similar means for allowing computer programs or other instructions to be loaded into ventilator 100. For example, memory 115 may include a removable memory chip (such as an EPROM, or PROM, or flash memory) and associated socket, and other removable storage units and interfaces which allow software and data to be transferred from a removable storage unit to ventilator 100. In some embodiments, memory 115 is a non-volatile memory. In some embodiments, memory 115 is configured for low-power consumption or configured to receive power only from an external source.

In some embodiments, control system 106 is coupled to power supply 108, which may be configured to provide power to the various components of ventilator 100. For example, control system 106 may be configured to route power from power supply 108 to motor 110 of blower 104. Power supply 108 may be disposed within medical device unit 102. Power supply 108 may include one or more of an internal rechargeable battery, a removable rechargeable battery, and a removable non-rechargeable battery. In some embodiments, control system 106 is coupled to power supply separate from power supply 108.

As shown in FIG. 4, medical device unit 102 may be configured to receive a battery pack via battery storage 137. In some embodiments, a user may place a removable rechargeable battery and/or a removable non-rechargeable battery within battery storage 137. In some embodiments, power supply 108 may be coupled to a power source (not shown) via a power adapter. Power supply 108 may control the voltage and current from a power source to control system 106.

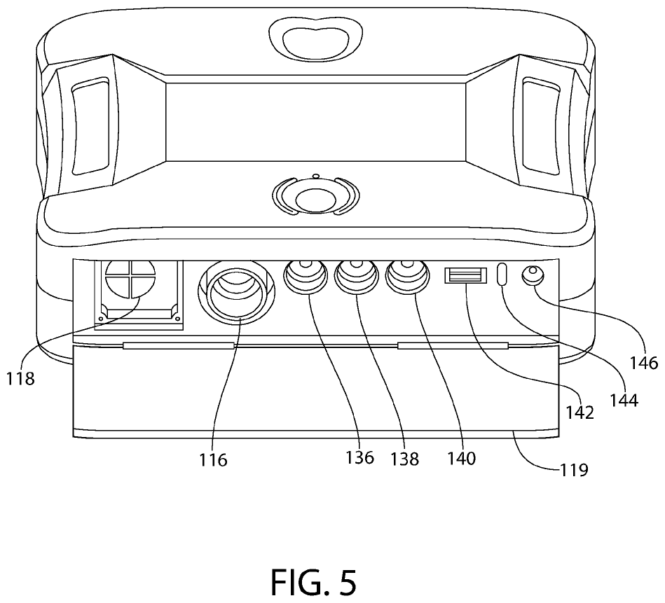

Referring to FIG. 5, in some embodiments, medical device unit 102 may include inlet 118 and outlet 116. Inlet 118 may be disposed on one of sidewalls 130 of housing 132 and allow for air to flow from the external environment (ambient air) or an air source, such as a reservoir of gas (O₂), to blower 104. For example, blower 104 may be configured to pull in air from inlet 118 and push the air out through outlet 116. In some embodiments, medical device unit 102 relies on blower 104 to provide air and does not require compressed air to operate. In some embodiments, blower 104 is coupled to outlet 116, which is disposed on an outer periphery of housing 132. For example, outlet 116 may be disposed on sidewall 130 of housing 132. Outlet 116 may be cylindrical in shape and hollow. In some embodiments, outlet 116 couples blower 104 to breathing circuit 200 to patient interface 300. For example, outlet 116 may be configured to allow air to flow from blower 104 of medical device unit 102 through breathing circuit 200 to patient interface 300. In some embodiments, outlet 116 is a valve that may open or close to control the airflow from blower 104 to breathing circuit 200. Outlet 116 may be controlled by air pressure or by control system 106.

Referring to FIGS. 1-2, ventilator 100 may include breathing circuit 200. Breathing circuit 200 may be coupled to medical device unit 102. For example, breathing circuit 200 may be coupled to outlet 116. In some embodiments, breathing circuit 200 may be disposed between medical device unit 102 and patient interface 300. Breathing circuit 200 may be configured to receive air from medical device unit 102. Breathing circuit 200 may include tube 202, exhale valve 208, flow sensor 210, and patient filter 212. Tube 202 may include first end 204 and second end 206. First end 204 may be coupled to medical device unit 102 and second end 206 may be coupled to patient interface 300. In some embodiments, tube 202 is a cylindrical lumen configured to allow airflow from medical device unit 102 to patient interface 300. Tube 202 may be configured to include exhale valve 208, flow sensor 210, and patient filter 212. Exhale valve 208 disposed on or within tube 202 and may be configured to open on exhalation of the patient using ventilator 100 to allow air to flow out of the patient. Exhale valve 208 may be closed during inhalation such that air does not exist ventilator 100, thereby increasing efficiency. For example, exhale valve 208 may be closed during inhalation to ensure that the proper amount and flow of air reaches patient interface 300.

In some embodiments, exhale valve 208 is controlled by control system 106 to control the exhalation of the patient. In another embodiment, exhale valve 208 is controlled based on the exhalation of the patient. In yet another embodiment, exhale valve is controlled by both control system 106 and the exhalation of the patient. Exhale valve 208 may be configured to allow for a specific respiration rate but may be opened by the exhalation of the patient as well. For example, for a respiration rate of 12 (one breathe ever five seconds), exhale valve 208 may open ever five seconds and may also open more than every five seconds if the patient is breathing at different rate.

In some embodiments, breathing circuit 200 includes flow sensor 210, which may be disposed on or within tube 202. Flow sensor 210 may be configured to sense the flow of air within breathing circuit 200. For example, flow sensor 210 may detect the rate and amount of air flowing through tube 202. In some embodiments, flow sensor 210 is coupled to control system 106 to provide feedback to ventilator 100. For example, flow sensor 210 may provide information to control system 106, which may change the parameters of blower 104 based on the information.

Breathing circuit 200 may further include patient filter 212, which may be disposed proximate second end 206 of tube 202. For example, patient filter 212 may be disposed on or within tube 202 proximate second end 206 and adjacent to patient interface 300. Patient filter 212 may be configured to filter out particles within air. For example, patient filter 212 may filter out particles and airborne viruses to protect the patient using ventilator 100.

Referring to FIGS. 1-2, ventilator 100 may include patient interface 300. Patient interface 300 may be a device that is secured to the face of a patient. For example, patient interface 300 may be a bag valve mask (e.g., mask 302), respirator, or an endotracheal (ET) tube used for intubation. In some embodiments, patient interface 300 is an ET tube inserted into a patient via intubation. Patient interface 300 may include mask 302 disposed over the mouth and/or nose of the patient. In some embodiments, mask 302 is coupled to medical device unit 102 via one or more tubes.

Referring to FIG. 5, medical device unit 102 may further included various inputs for coupling medical device unit 102 to other components of ventilator 100. In addition to inlet 118 and outlet 116, medical device unit 102 may include control line port 136, pressure line port 138, differential pressure tube port 140, flow sensor port 142, data communication port 144, and power port 146. Control line port 136 may be used to couple exhale valve 208 and medical device unit 102. For example, exhale valve 208 may be coupled to medical device unit 102 at control line port 136 such that medical device unit 102 can control the opening and closing of exhale valve 208. Pressure line port 138 and differential pressure tube port 140 may be used to couple one or more pressure sensors to medical device unit 102. Flow sensor port 142 may be used to couple flow sensor 210 to medical device unit 102. For example, flow sensor 210 may be coupled to medical device unit 102 at flow sensor port 142 such that medical device unit 102 can receive information from flow sensor 210. Data communication port 144 may be used to couple medical device unit 102 to an electronic device such as a computer system, a mobile device, a server, etc. Power port 146 may be used to couple medical device unit 102 to a power source. For example, power port 146 may be configured to couple power supply 108 to a power source to provide power to medical device unit 102 through power supply 108.

In some embodiments, ventilator 100 may be configured to administer a status check or a self-test to ensure that some or all of the components are working properly and that there are not any malfunctions. In response to the status check or self-check, ventilator 100 may receive a software update to address any errors or malfunctions detected by the status check or self-check. In some embodiments, ventilator 100 is configured to perform a status check or self-check without any human intervention and receives a software update based on the results of the status check or self-check. Ventilator 100 may transmit the results of the status check or self-check to a server, which may determine whether a software update is needed. In some embodiments, the results are reviewed by a user or a system (e.g., AI or machine learning system) to determine which components are malfunctioning and what updates are needed.

In some embodiments, control system 106 is configured to test the various components of ventilator 100 to determine the functional status of, for example, blower 104, power supply 108, writing device 113, memory 115, transmitting device 117, and control system 106, in addition to reporting the operational status of ventilator 100. For example, control system 106 may be configured to receive information from memory 115 regarding any corrupted cores, from blower 104 regarding an occlusion of fan 112, from outlet 116 or inlet 118 regarding occlusions, from power supply 108 regarding improper voltages, or any other information necessary to ensure that medical device unit 102 is functioning properly. Control system 106 may be configured to perform one or more self-tests on one or more components including a system start-up test, a motor test, a user interface test, a button test, a temperature sensor test, a motor voltage test, a motor current test, a motor initiation test, a patient pressure test, a blower pressure test, an oxygen sensor test, an ambient sensor test, a barometer test, a speaker test, a system fatal error test, and/or a software test. Control system 106 may be configured to receive a software update directed to the results of each and every one of the listed tests.

In some embodiments, control system 106 automatically receives information from various components of ventilator 100 on a periodic or aperiodic basis. For example, control system 106 may receive information for some or all of the components of medical device unit 102 without receiving a request from a user or other device. However, control system 106 may receive a request from a user to perform a self-test on one or more components of medical device unit 102.

In some embodiments, medical device unit 102 includes a wake-up controller. The wake-up controller may be communicatively coupled to control system 106. The wake-up controller may be configured to wake-up (e.g., power on) medical device unit 102 to allow control system 106 to perform a software update. In some embodiments, the wake-up controller is a microcontroller coupled to a timer. The timer may be configured to power on medical device unit 102 via the wake-up controller. The timer may transmit a signal to wake-up controller on a periodic basis, an aperiodic basis, a random basis, or a pre-programmed basis to power on medical device unit 102. The wake-up controller may include a clock or timer.

In some embodiments, the wake-up controller is configured to receive a request to perform a software update. Upon receiving the signal, wake-up controller may power on medical device unit 102 and instruct control system 106 to perform software update. In some embodiments, medical device unit 102 is powered on to download the software update. For example, medical device unit 102 may receive a request to download a software update. Medical device unit 102 may install the software update upon completion of the download or at another time (e.g., upon start-up of medical device unit 102 by a user).

In some embodiments, the software update is transmitted via a wireless connection (e.g., WiFi, Bluetooth, NFC, etc.) or a wired connection (e.g., ethernet connection or external device such as a USB or external computing device). Medical device unit 102 may be configured to be in a standby or sleep mode, such as during storage, and may install the software update upon receipt or when powered on. In some embodiments, during powering of medical device unit 102, a conformation for installation of the software update appears on user interface 124 and a user must interact with user interface 124 to confirm installation of the software update.

Medical device unit 102 may have a ventilation state or use state and a non-ventilation state or non-use state (e.g., a standby state and/or a powered off state). In the ventilation state, medical device unit 102 is actively providing ventilation (e.g., air flow/pressure) to a patient or is coupled to a patient to provide ventilation to the patient. In the non-ventilation state, medical device unit 102 is not actively providing ventilation, not coupled to a patient (e.g., patient interface 300) and/or is not in a ventilation mode. The non-ventilation state may include a standby state, where medical device unit 102 is powered on, but is not providing ventilation to a user, and a powered off state, where medical device unit 102 is not providing ventilation to a user and is not powered on.

In some embodiments, medical device unit 102 is configured to prevent installation of the software update during use (e.g., when medical device unit 102 is in the ventilation state). For example, while providing ventilation to a patient, medical device unit 102 may be configured to prevent installation of the software update to prevent medical device unit 102 from powering down or from performing incorrectly. In some embodiments, medical device unit 102 is configured to download the software update during use, but waiting until medical device unit 102 is not in used before installing the software update. Medical device unit 102 may have a default setting that while medical device unit 102 provides ventilation, a software update cannot be installed.

In some embodiments, wake-up controller and control system 106 are coupled to different power supplies. For example, wake-up controller may be coupled to power supply 108 (e.g., a battery) and control system 106 may be coupled to a different power supply and configured to run on a small amount of power. In some embodiments, control system 106 is coupled to a small battery configured to output little power and have longevity. Control system 106 may be coupled to a small battery due to performing a software update requiring low power consumption. In some embodiments, control system 106 and the wake-up controller are coupled to the same power supply. In some embodiments, wake-up controller is a low power controller. For example, wake-up controller may be a low power controller coupled to a power supply such that wake-up controller is configured to run for extended periods of time (e.g., several years).

In some embodiments, control system 106 is configured to perform a software update on a daily basis, weekly basis, monthly basis, bi-monthly basis, or any other amount of repeating time. Control system 106 may be configured to test certain components more frequently than other components. For example, control system 106 may be configured to test blower 104 on a monthly basis and speaker 141 on a daily basis to determine if a software update is needed. By way of another example, control system 106 may be configured to test all components sequentially or simultaneously on a monthly basis and test individual components (e.g., blower 104, speaker 141, user interface 124) on a weekly basis.

Control system 106 may communicate with the one or more failing components to determine the issue causing the failure and may transmit the results to a server to determine whether a software update is required. For example, upon indication that blower 104 is not functioning properly, control system 106 may communicate with one or more pressure sensors of blower 104 to determine whether there are errors with the pressure sensors (e.g., incorrect threshold values). In some embodiments, upon indication of a failure, control system 106 may cause speaker 141 to output an audio indication and/or cause user interface 124 to output a visual indication. The audio or video indication may present information to a user about malfunctioning components and/or whether a software update is required.

In some embodiments, medical device unit 102 of ventilator 100 is configured to perform a software update and then power down. In some embodiments, medical device unit 102 is configured to perform a software update while medical device unit 102 is in storage or otherwise not in active use (e.g., in a powered down state). In some embodiments, the software update is stored in memory 115. Medical device unit 102 may turn on without human intervention to install the software update. Alternatively, medical device unit 102 may install the software update upon start-up of medical device unit 102 by a user. For example, medical device unit 102 may power on, install a software update, store the software update within memory 115, and then power down. This allows medical device unit 102 to conserve power

In some embodiments, medical device unit 102 includes an accelerometer. Control system 106 may be configured to receive measurements from the accelerometer and may request readings from the accelerometer during a self-test. The accelerometer may indicate whether medical device unit 102 is moving, has been moved, or has been dropped. In some embodiments, control system 106 determines if measurement from the accelerometer exceeds a threshold value indicating that medical device unit 102 has been dropped and may be damaged. The accelerometer may also indicate whether medical device unit 102 has been moved and an indication of movement based on the measurements from the accelerometer may be included in the status data. In some embodiments, the accelerometer provides orientation of the medical device unit 102. Medical device unit 102 may include a gyroscope to provide orientation information to control system 106.

Medical device unit 102 may include an optical sensor (e.g., infrared sensor, passive infrared sensor). The optical sensor of medical device unit 102 may be coupled to control system 106, which may be coupled to user interface 124. Optical sensor may be configured to detect the amount of ambient light and decrease the brightness of user interface 124 based on the detected amount of ambient light to thereby decrease the power consumption of user interface 124. In some embodiments, optical sensor is configured to detect the presence of a user proximate to or viewing user interface 124. In the presence of a user, user interface 124 may display the status or status data of medical device unit 102. However, optical sensor may not detect a user or may detect the absence of a user proximate medical device unit 102 and may cause user interface 124 to not display the status or status data of medical device unit 102 since a user is not present to view user interface 124. This allows for a reduction in power consumption since user interface 124 does not need to be illuminated unless the optical sensor detects the presence of a user viewing user interface 124.

In some embodiments, medical device unit 102 includes one or more environmental sensors. The environmental sensors may include a temperature sensor, a barometric sensor, a gas sensor, and a humidity sensor. Medical device unit 102 may include a temperature sensor to measure the ambient temperature and/or internal temperature of medical device unit 102. In use, ambient temperature may affect the performance of one or more components of medical device unit 102, such as blower 104, pressure sensors, speaker 141, battery, or any other component of medical device unit 102. In some embodiments, changes to the ambient temperature detected by the temperature sensor results in changes to parameters of the self-test. For example, the pressure thresholds associated with blower 104 may vary based on the ambient temperature detected by the temperature sensor. In yet another example, the performance of the battery or power supply 108 may vary based on the reading by temperature sensor resulting in threshold levels of alarms for low battery levels varying. In some embodiments, medical device unit 102 outputs an alarm if temperature reading of temperature sensor deviates from a temperature range. The temperature range may be the range of temperatures that medical device unit 102 can adequately perform in. The temperature range may be from −50° C. to 50° C.

Medical device unit 102 may also include one or more gas sensors. For example, medical device unit 102 may include a first oxygen sensor for measuring the oxygen levels within medical device unit 102 due to leakage and a second oxygen sensor for measuring the oxygen concentration of gas/air delivered to a patient coupled to medical device unit 102 via breathing circuit 200. The first oxygen sensor may be configured to transmit an alert when the oxygen levels within medical device unit exceed a predetermined threshold. For example, due to fire hazard concerns, medical device unit 102 may prevent the buildup of air/gas within medical device unit 102 by allowing a small amount of air/gas within medical device unit 102 to leak out. Medical device unit 102 may vent out the oxygen when first oxygen sensor detects that the oxygen level within medical device unit 102 is above a pre-determined amount. For example, medical device unit 102 may be configured to vent or leak out air/oxygen when the first oxygen sensor determines that there is more than 15%, 20%, 25%, 30%, 35%, 40%, 45%, or 50% oxygen within medical device unit 102. In some embodiments, medical device unit 102 is configured to continuously vent or leak out air/oxygen to keep the amount of oxygen within medical device unit 102 at or below 15%, 20%, 25%, 30%, 35%, 40%, 45%, or 50%.

In some embodiments, the first oxygen sensor is an ambient oxygen sensor, and the second oxygen sensor is a galvanic oxygen sensor. The first oxygen sensor may be used to calibrate the second oxygen sensor. For example, the ambient oxygen sensor may be a highly sensitive oxygen sensor and may be used to calibrate the galvanic oxygen sensor, which may degrade overtime and during use such that it requires re-calibration. However, the second oxygen sensor may be used to calibrate the first oxygen sensor. In some embodiments, a signal oxygen sensor is used to measure the oxygen levels within medical device unit 102 and for measuring oxygen concentration of gas/air delivered to the patient.

Medical device unit 102 may include an altitude sensor, such as an altimeter or barometric pressure sensor. In some embodiments, controls system 106 receives measurements from the altitude sensor and transmits corrections to other sensors, such as the oxygen sensors. For example, control system 106 may determine that medical device unit 102 is at a higher altitude and thus the oxygen levels are lower compared to sea level. Control system 106 may calibrate the oxygen sensors based on the altitude measurements from the altitude sensor. For example, control system 106 may apply a correction factor to the oxygen sensors based on the altitude measurements from the altitude sensor. Medical device unit 102 may also include humidity sensor to detect the levels of ambient humidity. Medical device unit 102 may output an alarm or alert if the humidity levels detected by the humidity sensor exceed a predetermined threshold. For example, excess moisture, such as that detected by the humidity sensor, may cause damage to one or more components of medical device unit 102 or may one or more components of medical device unit 102 to perform incorrectly (e.g., gas sensors, battery, speaker 141).

In some embodiments, indicator 134 is configured to display or flash different colors of light based on the status of medical device unit 102. For example, indicator 134 may display or flash the color green when medical device unit 102 is operating normally, display or flash the color red when medical device unit 102 is malfunctioning, or display or flash the color yellow/orange when medical device unit 102 has an error, but can still function. Indicator 134 may flash a color or have a constant illumination. Indicator 134 may be any color desired and may alternate between different colors depending on the status of medical device unit 102. In some embodiments, indicator 134 is coupled to a beacon power supply to ensure that indicator 134 is able to continuously provide an indication for the status of medical device unit 102. The beacon power supply may be different than power supply 108.

Control system 106 may perform a software update without user intervention and may cause indicator 134 to illuminate based on the status of the software update (e.g., downloaded, installed, or failed). A user may view medical device unit 102 after the software update has been performed and may view indicator 134. Upon viewing indicator 134, a user may be able to determine the status of medical device unit 102 and if there are any errors associated with medical device unit 102 without having to interact with medical device unit 102. Interacting with medical device unit 102 may including actuating one or more buttons on medical device unit 102, powering on medical device unit 102, or engaging with user interface 124.

In practice, a user may view indicator 134 immediately after the software update has been performed or may view indicator 134 after a duration of time as elapsed since the software update has been performed. In some embodiments, control system 106 is configured to transmit a signal to indicator 134 regardless of the power status of medical device unit 102. In other words, indicator 134 may be configured to always receive a signal from control system 106 regardless of the power status of medical device unit 102. This may be due to control system 106 and indicator 134 each having their own power supply separate from power supply 108 or control system 106 and indicator 134 sharing a power supply separate from power supply 108. In some embodiments, indicator 134 has a low power sensor configured to receive a signal from control system 106 to illuminate based on the status (e.g., downloaded, installed, failed) of a software update.

Control system 106 may automatically test medical device unit 102 on a periodic, scheduled, aperiodic basis, or random basis to determine errors with one or more components of medical device unit 102. For example, control system 106 may power on medical device unit 102 and test all the components of medical device unit 102 on a periodic basis such as, for example, every month, every 3 months, or every 6 months. In response to the testing of the components, control system 106 may automatically transmit the results of the tests and receive a software update. Control system 106 may automatically download and install the software update

In some embodiments, a user may schedule specific dates for control system 106 to power on medical device unit 102, test one or more components of medical device unit 102, and perform a software update. For example, medical device unit 102 may be configured to have a programmable schedule to pre-schedule self-tests and software updates. Pre-scheduled self-tests and software updates may be used when large quantities of medical device units 102 are being transported for use such that defective, damaged, or inoperable medical device units 102 are identified prior to use. In another embodiment, control system 106 may power on medical device unit 102 and test all the components of medical device unit 102 on an aperiodic basis. For example, tests may need to run more frequently the longer medical device unit 102 is in storage. In some embodiments, control system 106 tests medical device unit 102 without user intervention.

In some embodiments, control system 106 is configured to autonomously power on medical device unit 102 to perform a software update. For example, control system 106 may be configured to periodically or aperiodically wake medical device unit 102 to perform a software update. In some embodiments, control system 106 performs software update in a “silent mode” such that a user is unable to notice that a software update is being performed. For example, control system 106 may download and install a software update without turning on user interface 124, speaker 141, indicator 133, or indicator 134 and without user intervention. In some embodiments, a user requests control system 106 to update the software controlling medical device unit 102 by interacting with user interface 124 or engaging with/actuating button 126.

In practice, medical device unit 102 may start-up/boot-up and perform a self-test, transmit the results of the self-test, and receive a software update in response to the self-test. The self-test may include testing of sensors (e.g., pressure sensors, oxygen sensors, temperature sensors, accelerometer, tachometer, gyroscope). In some embodiments, the self-test includes measuring the current drawn from one or more components (user interface 124, blower 104, speaker 141, control system 106) to determine whether the one or more components are receiving adequate current. The measurement of the current drawn by the one or more components may also determine if there is a short, cut wire, or detached wire. Once the status of one or more components of medical device unit 102 has been determined via the self-test, control system 106 may transmit a request for a software update and may receive a software update data package for download and installing.

Control system 106 may be configured to store and generate a history log based on previously performed software updates. In some embodiments, control system 106 is configured to store all previously installed software updates. Control system 106 may automatically transmit the history log on a periodic, aperiodic, or pre-scheduled basis. However, control system 106 may be configured to transmit the history log in response to a request.

In practice, multiple medical device units 102 may be stockpiled or placed in storage for a long period of time prior to use and thus may require multiple software updates to ensure that medical device unit 102 is functioning properly prior to use. In practice, previous medical devices require physical intervention (e.g., opening device, turning device on, etc.) to determine if the device is functioning properly. This is an inefficient use of resources and also depletes the devices power supply. In some embodiments, medical device unit 102 automatically powers on and performs a software update. Control system 106 of medical device unit 102 may store the software status data (e.g., downloaded, installed, failed) of the software update within memory 115. Writing device 113 may access the software status data from memory 115. In some embodiments, control system 106 sends the software status data directly to writing device 113. Writing device 113 may write the software status data to transmitting device 117, such as an RFID tag, which stores the software status data. After writing device 113 writes the software status data to transmitting device 117, medical device unit 102 may shut off to conserve power. A user may retrieve the software status data by using a receiving or reading device, such as an RFID reader, to wirelessly receive the software status data while medical device unit 102 is powered off.

In some embodiments, one transmitting device 117 may be associated with a plurality of medical device units 102. For example, a pallet or stockpile of medical device units 102 may be in communication with a single transmitting device 117 such that each writing device 113 of each medical device unit 102 writes the results of the software update (e.g., software status data) to the single transmitting device 117. This allows a user to determine whether medical device units 102 in a large volume of medical device units 102 are up to date on their software. This configuration allows for the monitoring and surveillance of a stockpile or large quantity of medical device units 102 without having to interact or be adjacent to each one. Further, this provides for a quick assessment of whether a stockpile or collection of medical device units 102 are ready for use.

In some embodiments, medical device unit 102 may include a wireless network module, such as a WiFi chip/card, configured to communicate with control system 106 and one or more external devices. The one or more external devices may include writing device 113, transmitting device 117, a server, a computer, a mobile device, or an external transmitter. The wireless network module may receive a signal from an external device causing medical device unit 102 to power on, administer a status check, transmit the results of the status check, and receive a software update to download and install. The software status data resulting from the software update on start-up may be stored within memory 115 and/or may be wirelessly transmitted to writing device 113, which may be disposed outside of medical device unit 102. Writing device 113 may then write the software status data to transmitting device 117, which may be stored within, on, or external to housing 132 of medical device unit 102. In some embodiments, writing device 113 and transmitting device 117 are each disposed proximate to medical device unit 102. In alternative embodiments, writing device 113 and transmitting device 117 are each disposed remote to medical device unit 102.

In some embodiments, medical device unit 102 may include speaker 141, additional lights, and/or additional display screens. Medical device unit 102 may be configured to alert a user regarding the status of the software update via one or more of user interface 124, indicator 133, indicator 134, user interface 124, display screens, or other modes of alerting a user. For example, medical device unit 102 may provide an alert, warning, or message to a user by text, audio, or visual indicators.

In some embodiments, controller 106 of medical device unit 102 in configured to execute one or more models (e.g., algorithms), such as a mathematical models, machine learning model, deep learning model, statistical model, etc., to provide an output to the user. The output may be presented on the user interface and/or may include instructions. In some embodiments, ventilator 100 utilizes artificial intelligence and/or machine learning to assess errors and provide rectifying instructions to a user (e.g., via a user interface).

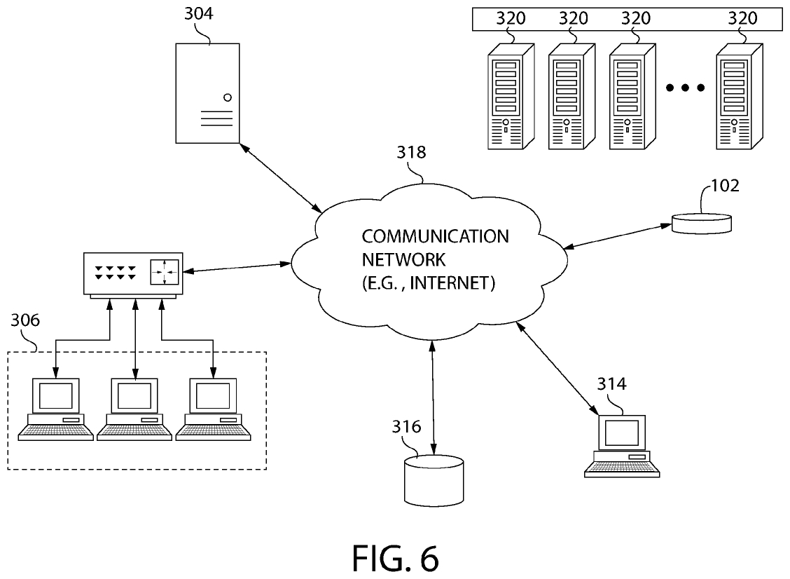

Referring to FIG. 7, medical device unit 102 may be configured to communicate with web server 304, cloud-based engine 321 including one or more processing devices 320, workstation 306, database 316, and one or more user computing devices 314 over network 318. For example, controller 106 of medical device unit 102 may be configured to communicate with one or more servers or devices over network 318. In some embodiments, medical device unit 102 communicates with one or more computing devices 314 over network 318. For example, a user may use a mobile device or computer to communicate with medical device unit 102 over network 318.

In some embodiments, medical device unit 102 assigns one or more the models (or parts thereof) for execution to one or more processing devices 320. For example, each model may be assigned to a virtual machine hosted by a processing device 320. The virtual machine may cause the models or parts thereof to execute on one or more processing units such as GPUs.

Medical device unit 102 may be configured to communicate with one or more model training systems that are communicatively coupled with at least one or more model database maintaining trained models and one or more training data databases (e.g., database 316) that stores relevant training data to train and/or retrain one or more models utilized by medical device unit 102. In some embodiments, medical device unit 102 communicates with server 304, which implements one or more models. The generated outputs of the one or more models may be transmitted to medical device unit 102. In some embodiments, medical device unit 102 outputs (e.g., via user interface 124) the generated outputs.

The model training system includes one or more model training servers or managers, which are implemented through one or more computing systems, servers, computers, processor and/or other such systems communicatively coupled with one or more of the distributed communication networks 318, and are configured to build and/or train the machine learning models. In some implementations, the model training system includes multiple sub-model training systems each associated with one or more of the different machine learning models.

In some embodiments, ventilator 100 utilizes one or more natural language processing (NLP) models to process spoken language. In some embodiments, the neural network, machine learning models and/or machine learning algorithms may include, but are not limited to, Large Language Models (LLM), Heuristics, Univariate based techniques, Multivariate, control limit, isolation forest and LOF—ensembles, deep learning models such as LSTM-based autoencoders, variational autoencoders, deep stacking networks (DSN), Tensor deep stacking networks, convolutional neural network, probabilistic neural network, autoencoder or Diabolo network, linear regression, support vector machine, Naïve Bayes, logistic regression, K-Nearest Neighbors (kNN), decision trees, random forest, gradient boosted decision trees (GBDT), K-Means Clustering, hierarchical clustering, DBSCAN clustering, principal component analysis (PCA), and/or other such models, networks and/or algorithms.

In some embodiments, ventilator 100 utilizes models (e.g., machine learning models) to provide instructions or responses to a user. For example, ventilator 100 may indicate that an error has occurred (e.g., an obstruction) and ventilator 100 may use one or more models to provide rectification instructions. The one or more models may be trained based on collective data obtained from a plurality of ventilators 100. In some embodiments, the one or more models are trained based data acquired from the plurality of ventilators 100. For example, a user may clear the obstruction using a series of inputs to ventilator 100. The series of inputs may be logged within database (e.g., database 316) and used to train one or more models to provide instructions to a user for clearing a similarly detected obstruction.

In some embodiments, ventilator 100 utilizes one or more models to provide guidance to a user of ventilator 100. For example, based on historical data, one or more models may be trained to identify or predict errors and issues and provide instructions to the user to prevent the errors or issues from occurring.

In some embodiments, ventilator 100 is configured to provide instructions or guidance to a user on using ventilator 100 to provide resuscitation to a patient. Ventilator 100 may provide step-by-step instructions and adjust the instructions based on prompts from the user or signal received from various sensor communicating with controller 106. For example, ventilator 100 may provide audio and/or visual instructions and guidance to allow a user to interact with a patient without having to continuously interact with ventilator 100. Ventilator 100 may utilize NLP to receive audio prompts, input from the user, and provide responses to allow the user to interact with a patient while simultaneously interacting with ventilator 100. In some embodiments, a user may interact with ventilator 100 without manually or physically interacting with user interface 124. For example, controller 106 may be configured to parse and extract keywords associated with the operation of medical device unit 102. Controller 106, based on the parsed and extracted keywords, may identify a request by the user and provide instructions (e.g., an audio output) to the user in response to the request.

In some embodiments, each ventilator 100 logs inputs, prompts, and actions taken (e.g., activity data) and transmits the activity data to one or more databases (e.g., database 316). One or more models may be trained based on the activity data and may generate outputs or instructions for rectifying errors or issues that have occurred with ventilator 100. Ventilator 100 may then receive a remote update including the generated outputs or instructions such that each ventilator 100 remains up to date with the newest rectifying instructions.

In some embodiments, ventilator 100 keeps track of how errors and issues are addressed during use (e.g., error data). Ventilator 100 may transmit the error data to database 316. In some embodiments, one or more models are trained using the error data to generate rectifying instructions. The rectifying instructions may provide instructions to a user on how to address and rectify one or more errors or issues that arise during use of ventilator 100. The rectifying instructions generated by the one or more models may be transmitted to ventilator 100 via a remote update.

In some embodiments, ventilator 100 is configured to communicate with adjacent ventilators 100. For example, a first ventilator may be configured to communicate with a second ventilator (e.g., over network 318). The first ventilator may receive a remote software update and may transmit the remote software update to the second ventilator. This allows a single ventilator to spread the remote software update to one or more adjacent ventilators without requiring the one or more adjacent ventilators from having to communicate with the server (e.g., web server 304) to receive the remote software update. For example, a plurality of ventilators 100 may be stored in a stockpile and at least one ventilator 100 may be coupled to the server to receive a remote software update. The at least one ventilator 100 may be configured to receive, download, and install the remote software update and then transmit the remote software update to the other ventilators 100 within the stockpile. In some embodiments, the at least one ventilator 100 that initially receives the remote software update is configured to install the software update and check for errors prior to transmitting the software update to adjacent ventilators 100. If an error is detected (e.g., indicating that the software update is corrupted), the at least one ventilator 100 does not transmit the software update to adjacent ventilators 100 until the error is rectified. This prevents a large number of ventilators 100 from being infected with a corrupted software update. Allowing a single ventilator 100 to transmit a software update to adjacent ventilators 100 allows many ventilators 100 to be updated at once and also allows for patching of software updates. Further, this allows many ventilators 100 to be updated without having all of them communicatively coupled to a server. This also allows a single ventilator 100 to fix or rectify a plurality of adjacent ventilators that have errors or corrupted software updates.

In some embodiments, ventilator 100 is configured to continuously poll for a remote software update. Alternatively, ventilator 100 may be configured to passively wait until a push command for a remote software update is received. For example, a plurality of ventilators 100 may be stored within a stockpile and a user may use a transmitting device to push a software update to one or more ventilators 100 in the stockpile. Ventilators 100 may be configured to receive the software update based on the pushed request.

In some embodiments, ventilator 100 is configured to provide autonomous or semi-autonomous clinical management to a patient. For example, once the patient is connected to ventilator 100, ventilator 100 may be configured to detect vital signs (e.g., pulse, oxygen saturation, respiration rate) and automatically adjust and configure ventilation parameters to provide ventilation to the patient. Ventilator 100 may be configured to adjust ventilation parameters without human intervention based on the detected vital signs. In some embodiments, ventilator 100 is configured to adjust ventilation parameters based on industry standard guidelines (e.g., American Heart Association guidelines). Ventilator 100 may receive remote software updates, which may include any changes to the industry standard guidelines that have been enacted.

In some embodiments, a user can manually adjust settings and parameters of ventilator 100. Ventilator 100 may have constraints on the upper and lower limits of the settings and parameters based on industry standard guidelines. In some embodiments, ventilator 100 receives a remote software update including changes or revisions to the industry standard guidelines and automatically adjusts the constraints of the settings and parameters of ventilator 100.

In some embodiments, upon completing a self-test, ventilator 100 transmits the results of the self-test to a server (e.g., web server 304). The self-test may indicate changes in tolerances of one or more components. In some embodiments, the changes in tolerances of the one or more components are within an acceptable range. However, when the changes in tolerances of the one or more components are not within an acceptable range, the server may transmit a remote software update to adjust the one or more components such that the tolerances are within the acceptable range.

In some embodiments, the remote software update is configured to add additional features as they become available and/or are approved by regulatory panels. For example, ventilator 100 may include the necessary hardware, but may need adjustments to software to execute specific features. In some embodiments, ventilator 100 includes one or more components that are restricted or blocked from normal use. The one or more components may be accessible and/or unblocked upon receiving a remote software update. For example, ventilator 100 may include a blower (e.g., blower 104) having a fan (e.g., fan 112) that is initially limited to a maximum RPM of 20,000. Upon receiving a remote software update, the maximum RPM of the fan may be increased to greater than 20,000 RPM, such as 37,500 RPM.

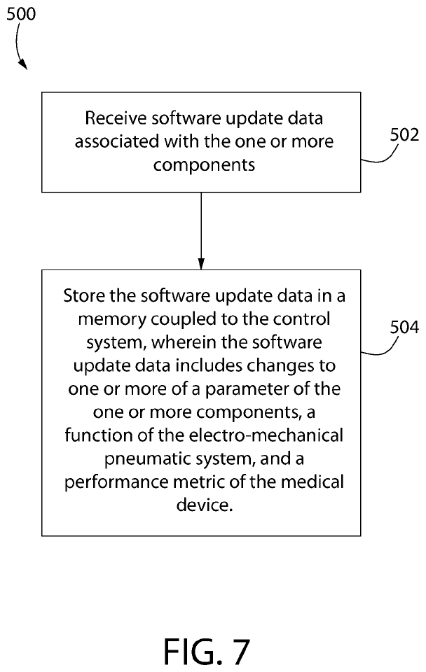

In some embodiments, ventilator 100 includes one or more cameras, microphones, and/or sensors configured to receive audio and/or video input. The camera, microphone, and/or sensors may be configured to receive and record audio and/or video. Ventilator 100 may utilize one or more models implementing machine vision to watch and/or listen to the user interact with the patent in real-time and provide instructions, as necessary. For example, ventilator 100 may include a camera and sensors that detect the motion of the user as the user interacts with the patient. Using one or more models implementing machine vision, ventilator 100 may detect that the user has connected ventilator 100 to the patient incorrectly resulting in a leak of oxygen. Ventilator 100 may provide instructions (e.g., video and/or audio) to the user to rectify the detected error. FIG. 8 illustrates a flow diagram of an exemplary method 500 used by ventilator 100 to providing a software update to a medical device. Method 500 may include step 502 of receiving, without human intervention, software update data associated with the one or more components. Method 500 may also include step 504 of storing the software update data in a memory coupled to the control system, wherein the software update data includes changes to a parameter of the one or more components.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

What is claimed is:

1. A medical device comprising:
an electro-mechanical pneumatic assembly configured to generate air flow, the electro-mechanical pneumatic assembly disposed within the medical device and coupled to one or more components; and
a controller coupled to the electro-mechanical pneumatic assembly, the controller configured to:
receive software update data associated with the one or more components; and
store the software update data in a memory coupled to the controller, wherein the software update data includes changes to one or more of a parameter of the one or more components, a function of the electro-mechanical pneumatic assembly, and a performance metric of the medical device.

2. The medical device of claim 1 further comprising:
a beacon configured to provide an indication representative of the software update data.

3. The medical device of claim 2, wherein the beacon is configured to request the software update.

4. The medical device of claim 2, wherein the beacon is configured to determine an availability of the software update.

5. The medical device of claim 2, wherein the beacon transmits a software version of the medical device to a server on a periodic basis and the server compares the software version to the software update and transmits the software update to the medical device.

6. The medical device of claim 1, wherein the controller receives the software update without human intervention.

7. The medical device of claim 1, wherein the controller is configured to output a signal to one or more of a speaker, a user interface, a storage device, an external device, a beacon, a writing device, a transmitting device, and an indicator.

8. The medical device of claim 1 further comprising:
a wake-up controller coupled to the controller, the wake-up controller configured to power on the medical device prior to the controller receiving the software update data.

9. The medical device of claim 1, wherein the controller is further configured to:
display on a user interface, via a display screen or a light indicator, a software status associated with the software update data.

10. The medical device of claim 1, wherein the controller includes a low power controller configured to receive the software update data.

11. The medical device of claim 1, wherein the controller is further configured to:
receive plurality of inputs from a user, the plurality of inputs associated with operation of the medical device;
store the plurality of inputs in a database; and
receive subsequent software update data including instruction data associated with the plurality of inputs.

12. The medical device of claim 1, wherein the controller is further configured to:
receive, from a user, a plurality of audio prompts;
use one or more models, parse and extract from the audio prompts one or more keywords associated with a request; and
output an audio response based on the request.

13. A method of providing a software update to a medical device, the method comprising:
receiving, software update data associated with one or more components of a ventilator, the ventilator including an electro-mechanical pneumatic assembly disposed within the ventilator and coupled to the one or more components; and
storing the software update data in a memory coupled to a controller, the controller coupled to the electro-mechanical pneumatic assembly,
wherein the software update data includes changes to a parameter of the one or more components.

14. The method of claim 13 further comprising:
receiving a plurality of inputs from a user, the plurality of inputs associated with operation of the medical device;
storing the plurality of inputs in a database; and
receiving subsequent software update data including instruction data associated with the plurality of inputs.

15. The method of claim 13 further comprising:
receiving, from a user, a plurality of audio prompts;
using one or more models, parse and extract from the audio prompts one or more keywords associated with a request; and
outputting an audio response based on the request.

16. The method of claim 13 further comprising:
powering on the medical device, via a wake-up controller coupled to the controller, prior to the controller receiving the software update data.

17. The method of claim 13 further comprising:
displaying on a user interface, via a display screen or a light indicator, a software status associated with the software update data.

18. The method of claim 13 further comprising:
determining that the medical device has transitioned from use state to a non-use state; and
retrieving the software update data from the memory for installation in response to the determination that the medical device is the non-use state.

19. A method of providing a software update to a medical device, the method comprising:
receiving software update data associated with a ventilator, the ventilator including an electro-mechanical pneumatic assembly coupled to one or more components;
storing the software update data in a memory coupled to a controller, the controller coupled to the electro-mechanical pneumatic assembly;
determining that the ventilator is in a ventilation state, the ventilation state being when the electro-mechanical pneumatic assembly is providing ventilation to a patient; and
preventing retrieval of the software update data from the memory for installation in response to the determination that the ventilator is in the ventilation state.

20. The method of claim 19 further comprising:
determining that the ventilator has transitioned from the ventilation state to a non-ventilation state; and
retrieving the software update data from the memory for installation in response to the determination that the ventilator is the non-ventilation state.
